(19) United States
(12) Patent Application Publication
(10) Pub. No.: US 2024/0349810 A1
(43) Pub. Date: Oct. 24, 2024

(54) ATOMIZER AND ELECTRONIC ATOMIZATION DEVICE

(71) Applicant: Jiangmen Moore Technology., Ltd, Jiangmen (CN)

(72) Inventors: Boxue GONG, Jiangmen (CN); Yueyang ZHAO, Jiangmen (CN); Ju XIE, Jiangmen (CN); Wenyuan FAN, Jiangmen (CN); Zhenyu WU, Jiangmen (CN); Ming LYU, Jiangmen (CN); Guoliang OU, Jiangmen (CN)

(21) Appl. No.: 18/759,073

(22) Filed: Jun. 28, 2024

Related U.S. Application Data
(63) Continuation of application No. PCT/CN2022/117635, filed on Sep. 7, 2022.

(30) Foreign Application Priority Data
Dec. 30, 2021 (CN) 202123432678.1

(51) Int. Cl. A24F 40/48; A24F 40/10; A24F 40/42; A24F 40/46

(52) U.S. Cl. CPC A24F 40/48; A24F 40/10; A24F 40/42; A24F 40/46

(57) ABSTRACT

An atomizer includes: a housing provided with an accommodating cavity and an air outlet channel; an atomization base with at least a partial structure disposed in the accommodating cavity, a liquid storage cavity for storing an aerosol-generation substrate being defined between a top wall of the atomization base and the housing, the atomization base including an atomization cavity and at least one liquid flowing channel, the liquid flowing channel being communicated between the liquid storage cavity and the atomization cavity; and a vent channel provided with a vent outlet, the vent outlet being communicated with the liquid storage cavity and being disposed close to the air outlet channel.

### CROSS-REFERENCE TO PRIOR APPLICATION

This application is a continuation of International Patent Application No. PCT/CN2022/117635, filed on Sep. 7, 2022, which claims priority to Chinese Patent Application No. 202123432678.1, filed on Dec. 30, 2021. The entire disclosure of both applications is hereby incorporated by reference herein.

### FIELD

This application relates to the field of atomization technology, and more specifically, to an atomizer and an electronic atomization device.

### BACKGROUND

An electronic atomization device usually includes an atomizer and a power supply component. The power supply component is configured to supply power to the atomizer. The atomizer converts electric energy into thermal energy. Under the action of the thermal energy, an aerosol-generation substrate is converted into an aerosol for a user to inhale. During inhalation, a liquid level in a liquid storage cavity decreases, an air pressure is reduced, and air needs to be supplemented through a vent channel, or otherwise the flowing of liquid is affected. In a process in which external air enters the liquid storage cavity through an atomization core or a vent structure, the aerosol-generation substrate with high viscosity blocks a gas from flowing upward, forming bubbles with different sizes. When there are too many bubbles or excessively large bubbles, bubbles tend to get stuck in a liquid flowing channel, and as a result the liquid suction of the atomization core is hindered. The atomization core performs dry heating due to unsmooth liquid flowing, affecting the service life of the atomizer and the user experience of a user.

### SUMMARY

In an embodiment, the present invention provides an atomizer, comprising: a housing provided with an accommodating cavity and an air outlet channel; an atomization base with at least a partial structure disposed in the accommodating cavity, a liquid storage cavity configured to store an aerosol-generation substrate being defined between a top wall of the atomization base and the housing, the atomization base including an atomization cavity and at least one liquid flowing channel, the liquid flowing channel being communicated between the liquid storage cavity and the atomization cavity; and a vent channel provided with a vent outlet, the vent outlet being communicated with the liquid storage cavity and being disposed close to the air outlet channel.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an electronic atomization device according to an embodiment of this application;

FIG. 2 is a cross-sectional view of FIG. 1;

FIG. 3 is an exploded view of FIG. 1;

FIG. 4 is a schematic structural diagram of a top atomization base shown in FIG. 2;

FIG. 5 is a cross-sectional view of FIG. 4;

FIG. 6 is a schematic structural diagram of an atomization base shown in FIG. 2;

FIG. 7 is a schematic structural diagram of an atomization base shown in FIG. 2 from another viewing angle;

FIG. 8 is a schematic structural diagram of an atomization base shown in FIG. 2 from still another viewing angle;

FIG. 9 is a schematic structural diagram of an atomization base provided with a first seal member shown in FIG. 2;

FIG. 10 is a schematic structural diagram of an electronic atomization device according to another embodiment of this application;

FIG. 11 is a cross-sectional view of FIG. 10;

FIG. 12 is an exploded view of FIG. 10;

FIG. 13 is a schematic structural diagram of a top atomization base shown in FIG. 11;

FIG. 14 is a schematic structural diagram of a top atomization base shown in FIG. 11 from another viewing angle;

FIG. 15 is a cross-sectional view of FIG. 13;

FIG. 16 is a schematic structural diagram of a bottom atomization base shown in FIG. 11;

FIG. 17 is a schematic structural diagram of an atomization base shown in FIG. 11;

FIG. 18 is a schematic structural diagram of a top atomization base in an implementation of an embodiment of this application from a viewing angle;

FIG. 19 is a schematic structural diagram of a top atomization base in an implementation of an embodiment of this application from another viewing angle;

FIG. 20 is a schematic structural cross-sectional view of a top atomization base in an implementation of an embodiment of this application from a viewing angle;

FIG. 21 is a schematic structural cross-sectional view of a top atomization base in an implementation of an embodiment of this application from another viewing angle;

FIG. 22 is a schematic structural diagram of an atomizer in an implementation of an embodiment of this application;

FIG. 23 is a schematic structural exploded view of an atomizer in an implementation of an embodiment of this application;

FIG. 24 is a schematic structural cross-sectional view of an atomizer in an implementation of an embodiment of this application from a viewing angle;

FIG. 25 is a schematic structural cross-sectional view of an atomizer in an implementation of an embodiment of this application from another viewing angle;

FIG. 26 is a partial schematic structural enlarged view of FIG. 25;

FIG. 27 is a schematic structural cross-sectional view of an atomizer in an implementation of an embodiment of this application from still another viewing angle;

FIG. 28 is a schematic structural diagram of an electronic atomization device in an implementation of an embodiment of this application;

FIG. 29 is a partial schematic structural exploded view of an electronic atomization device in an implementation of an embodiment of this application; and

FIG. 30 is a schematic structural cross-sectional view of an electronic atomization device in an implementation of an embodiment of this application.

### DETAILED DESCRIPTION

In an embodiment, the present invention provides an atomizer and an electronic atomization device, to mitigate a case that bubbles get stuck in a liquid flowing channel, thereby improving the service life of the atomizer and the user experience of a user.

In an embodiment, the present invention provides an atomizer, including:
a housing, where the housing is provided with an accommodating cavity and an air outlet channel;
an atomization base with at least a partial structure disposed in the accommodating cavity, where a liquid storage cavity configured to store an aerosol-generation substrate is defined between the top wall of the atomization base and the housing, the atomization base is formed with an atomization cavity and at least one liquid flowing channel, and the liquid flowing channel is communicated between the liquid storage cavity and the atomization cavity; and
a vent channel provided with a vent outlet, where the vent outlet is communicated with the liquid storage cavity, and is disposed close to the air outlet channel.

In an implementation, the atomizer includes an atomization core disposed in the atomization cavity, and the aerosol-generation substrate in the liquid storage cavity is guided to the atomization core through the liquid flowing channel; and the atomization core includes a heating body, the vent channel having a vent inlet provided in the atomization cavity, and the vent inlet is disposed in a region of the atomization base located at the periphery of the heating body.

In an implementation, a plurality of vent channels are provided, and the vent channels are symmetrically distributed along the central axis of the air outlet channel.

In an implementation, a plurality of liquid flowing channels are provided, and the liquid flowing channels are symmetrically distributed along the central axis of the air outlet channel.

In an implementation, an air guide channel and a vent opening are opened in the atomization base, the air guide channel includes an open end and a closed end opposite to the open end, the vent opening is located across two sides of the central axis of the air guide channel in a first direction, and the air guide channel is communicated with the atomization cavity through the vent opening and is communicated with the air outlet channel through the open end, where
the first direction is perpendicular to the central axis of the air guide channel.

In an implementation, the vent outlet is provided in at least one side of the air guide channel in the first direction.

In an implementation, the atomization base includes a bottom atomization base and a top atomization base, the atomization cavity is defined between the bottom atomization base and the top atomization base, and the air guide channel, the vent opening, and the liquid flowing channel are opened in the top atomization base.

In an implementation, the top atomization base includes:
a top base body, where the top base body includes a top wall and a side wall surrounding the top wall, the top wall and the side wall surround to form the atomization cavity that is independent of the liquid storage cavity and provided with an opening at an end, and the liquid flowing channel is provided in the top wall; and
a boss disposed protruding from the top wall, where the air guide channel is formed in the boss, and the vent outlet is formed in the boss and is provided close to the open end.

In an implementation, the peripheral wall of the top base body is formed with a first vent groove communicated with the vent outlet, and the end of the first vent groove away from the vent outlet is communicated with the atomization cavity.

In an implementation, the first vent groove includes a first sub-groove communicated with the vent outlet and a second sub-groove communicated with the atomization cavity, the first sub-groove is provided in the top wall and the peripheral wall of the boss, and the second sub-groove is provided in the peripheral wall of the side wall; and
the end of the first sub-groove away from the vent outlet is communicated with the second sub-groove; or, the peripheral wall of the bottom atomization base is formed with a second vent groove, and the end of the first sub-groove away from the vent outlet is communicated with the second sub-groove through the second vent groove.

In an implementation, a plurality of second vent grooves are provided, and the second vent grooves are sequentially communicated.

In an implementation, the peripheral wall of the bottom atomization base is formed with the first vent groove communicating the second vent grooves.

In an implementation, a plurality of second sub-grooves are provided, and the second sub-grooves are joined to and communicated with the corresponding second vent grooves.

In an implementation, the atomizer further includes a first seal member, and the first seal member is disposed at the top end of the top atomization base; and
one part of the vent channel is defined between the first seal member and the groove wall of the first sub-groove, and the other part of the vent channel is defined between the side wall of the accommodating cavity and the groove walls of the second sub-groove and the second vent groove, where
a first vent hole is further opened in the first seal member, and the first vent hole is communicated between the liquid storage cavity and the vent outlet.

In an implementation, a liquid inlet opening of the liquid flowing channel, the open end, and the vent outlet are all formed at the top end of the top atomization base.

In an implementation, the top atomization base is formed with a third vent groove communicated with the vent outlet; and the end of the third vent groove away from the vent outlet is communicated with the atomization cavity.

In an implementation, the bottom atomization base is formed with a fourth vent groove communicated with the atomization cavity, and the end of the third vent groove away from the vent outlet is communicated with the atomization cavity through the fourth vent groove.

In an implementation, the third vent groove includes a plurality of third sub-grooves formed in the peripheral wall of the top atomization base, and the third sub-grooves are sequentially communicated, or, the peripheral wall of the top atomization base is formed with a second vent groove communicating the third sub-grooves; and
the fourth vent groove is communicated with the first third sub-groove in an airflow flowing direction, and the vent outlet is communicated with the last third sub-groove in the airflow flowing direction.

In an implementation, the bottom atomization base includes a body and a connection portion disposed protruding from the body, the bottom portion of the top atomization base abuts against the body, and the peripheral wall of the top atomization base is clamped at the connection portion.

In an implementation, the side wall of the atomization cavity forms one part of the fourth vent groove, the top end of the body forms the other part of the fourth vent groove, the third vent groove includes a fourth sub-groove extending inward along the peripheral wall of the top atomization base, and the fourth vent groove is communicated with the first third sub-groove in the airflow flowing direction through the fourth sub-groove.

In an implementation, the atomizer further includes a second seal member, and the second seal member is disposed at the top end of the top atomization base; and
one part of the vent channel is defined between the second seal member and the groove walls of the third sub-grooves, and the other part of the vent channel is defined between the top atomization base and the bottom atomization base, where
a second vent hole is further opened in the second seal member, and the second vent hole is communicated between the liquid storage cavity and the vent outlet.

Embodiments of this application further provide an electronic atomization device, including the foregoing atomizer. The atomizer provided in the embodiments of this application, the vent channel is provided. The vent outlet of the vent channel is communicated with the liquid storage cavity. The aerosol-generation substrate in the liquid storage cavity is guided into the atomization cavity through the liquid flowing channel for heating and atomization to generate an aerosol. The aerosol passes through the air outlet channel for a user to inhale. After the aerosol-generation substrate in the liquid storage cavity is consumed, external air enters the liquid storage cavity through the vent channel to balance a pressure in the liquid storage cavity. When flowing through the air outlet channel, the high-temperature aerosol generated from the heating and atomization exchanges heat with the side wall of the air outlet channel. For example, the high-temperature aerosol performs convective heat exchange, condensing heat release, and the like. Therefore, the temperature of the side wall of the air outlet channel is increased, and the side wall of the air outlet channel conducts heat to the aerosol-generation substrate near the air outlet channel. The aerosol-generation substrate near the air outlet channel absorbs heat, undergoes a temperature rise, and has reduced viscosity. In this way, the vent outlet is provided close to the air outlet channel, and bubbles generated at the vent outlet form and spread in a region of the aerosol-generation substrate with low viscosity. That is, the vent outlet is provided close to the air outlet channel. In one aspect, the aerosol-generation substrate near the vent outlet may have low viscosity. This makes it easier for air to enter the liquid storage cavity through the vent channel, so that while ventilation is improved, the vent channel may be kept from being blocked. In another aspect, the aerosol-generation substrate near the air outlet channel absorbs heat, undergoes a temperature rise, and has reduced viscosity. This makes it convenient for bubbles generated at the vent outlet to spread in an extension direction of the air outlet channel, so that bubbles generated at the vent outlet can be kept from spreading transversely and from blocking the liquid flowing channel. In this way, a case that bubbles get stuck in the liquid flowing channel can be mitigated, thereby improving the service life of the atomizer and the user experience of a user.

It is to be noted that the embodiments in this application and the technical features in the embodiments may be combined with each other without causing any conflict, and the detailed description in specific implementations should be understood as an explanation and description of the purpose of this application, but should not be considered as an inappropriate limitation to this application.

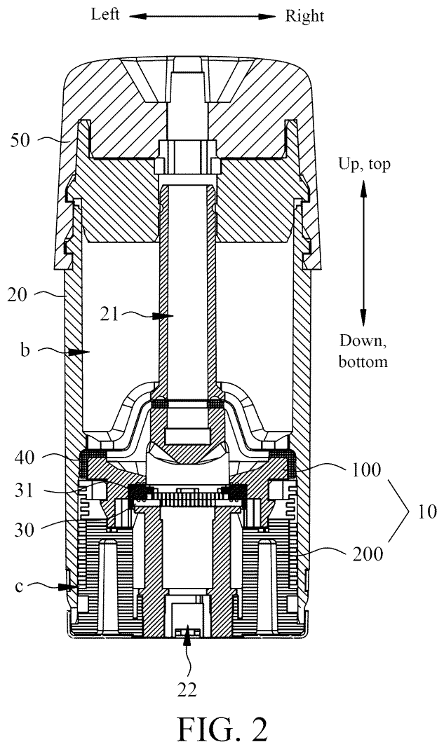

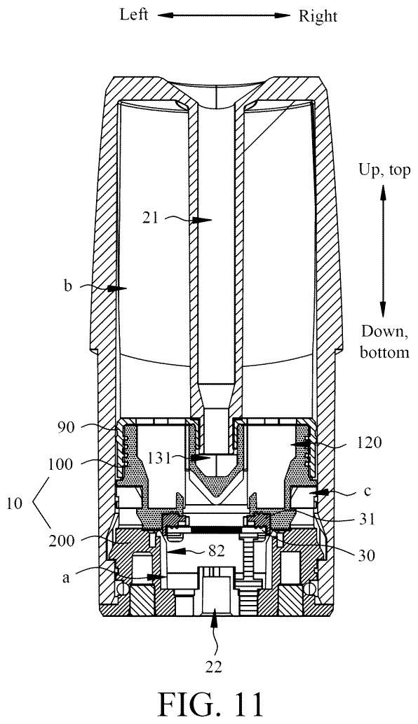

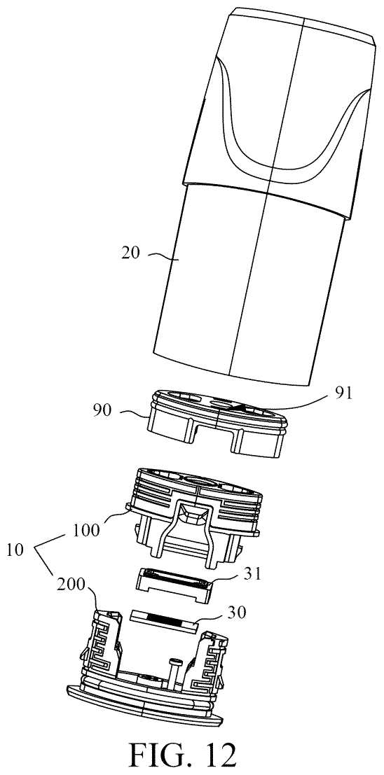

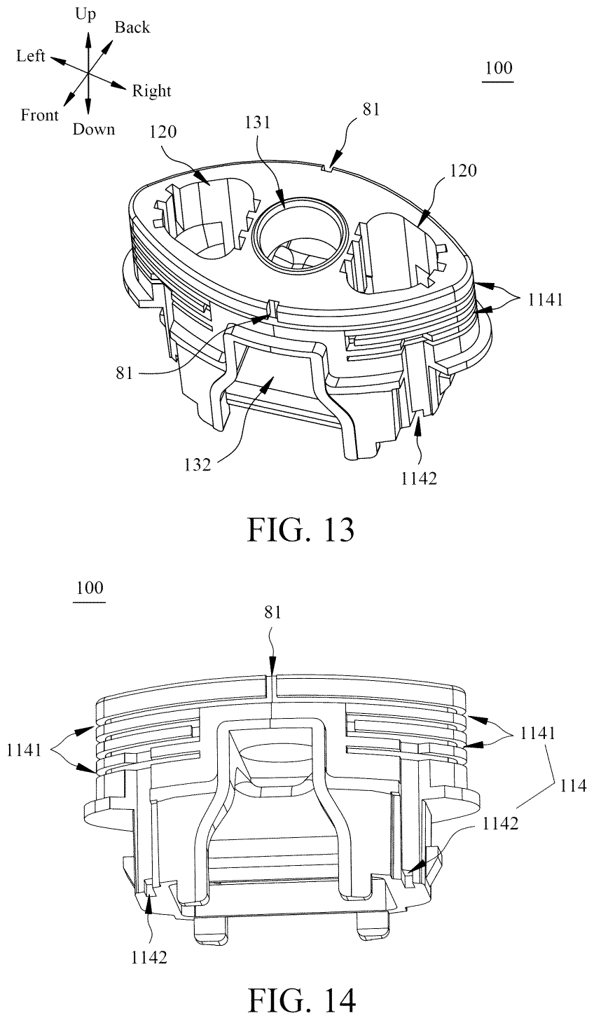

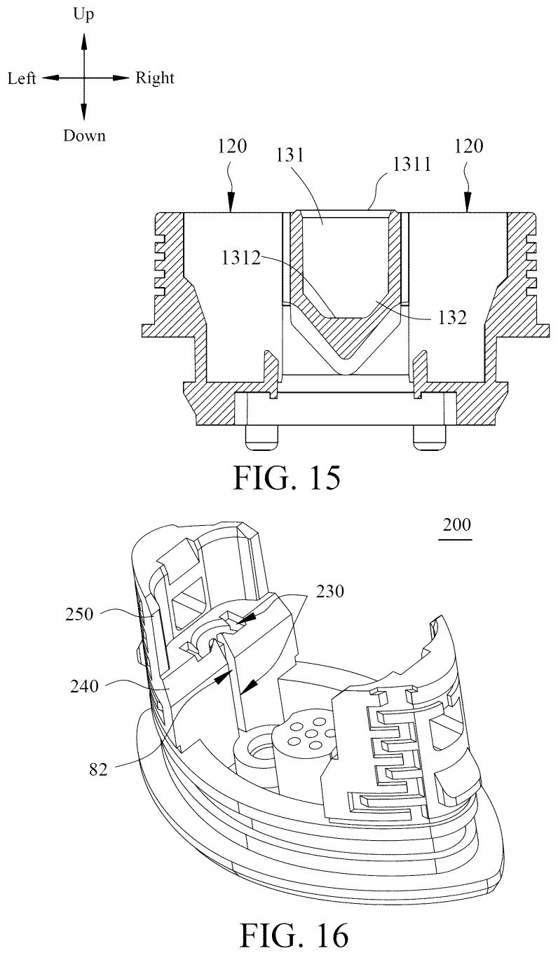

In the embodiments of this application, orientation or position relationships indicated by “up”, “down”, “left”, “right”, “front”, “back”, “top”, and “bottom” are based on orientation or position relationships based on FIG. 2, FIG. 5, FIG. 11, FIG. 13, and FIG. 15. A “height” is a top-bottom direction based on FIG. 5 and FIG. 15. A “first direction” is a front-back direction based on FIG. 13. A “second direction” is a left-right direction based on FIG. 13. It is to be understood that these orientation terms are used only for case and brevity of illustration and description in this application, rather than indicating or implying that the mentioned device or component needs to have a particular orientation or needs to be constructed and operated in a particular orientation. Therefore, such terms are not to be construed as limiting of this application. This application is further described below in detail with reference to the accompanying drawings and specific embodiments.

It may be understood that the terms “first”, “second”, “third”, “fourth”, and the like used in this application may be used herein to describe various terms, and shall not be construed as indicating or implying relative importance or implying the quantity of indicated technical features. However, unless specifically described, these terms are not limited by these terms. These terms are only used for distinguishing one term from another term. For example, without departing from the scope of this application, a first liquid inlet opening and a second liquid inlet opening are different liquid inlet openings, a first central axis, a second central axis, a third central axis, and a fourth central axis are different central axes, a first corner and a second corner are different corners, a first plane and a second plane are different planes, and a first gap channel and a second gap channel are different gap channels. In the description of the embodiments of this application, “a plurality of” and “several” means at least two, such as two and three unless it is specifically defined otherwise.

In the description of the embodiments of this application, unless otherwise explicitly specified or defined, the terms such as “mount”, “install”, “connect”, “connection”, and “fixed” should be understood in a broad sense. For example, the connection may be a fixed connection, a detachable connection, or an integral connection; or the connection may be a mechanical connection or an electrical connection; or the connection may be a direct connection, an indirect connection through an intermediary, or internal communication between two components or mutual interaction relationship between two components unless otherwise explicitly defined. A person of ordinary skill in the art may understand the specific meanings of the foregoing terms in the embodiments of this application according to specific situations.

In the description of the embodiments of this application, unless otherwise explicitly specified or defined, the first feature being located “above” or “below” the second feature may be the first feature being in a direct contact with the second feature, or the first feature being in an indirect contact with the second feature through an intermediary. In addition, that the first feature is “above”, “over”, or “on” the second feature may indicate that the first feature is directly above or obliquely above the second feature, or may merely indicate that the horizontal position of the first feature is higher than that of the second feature. That the first feature is “below”, “under”, and “beneath” the second feature may be that the first feature is right below the second feature or at an inclined bottom of the second feature, or may merely indicate that the horizontal position of the first feature is lower than that of the second feature.

It should be noted that, when a component is referred to as “being fixed to” or “being disposed at” another component, the component may be directly on the another component, or an intermediate component may exist. When one component is considered as “being connected to” another component, the component may be directly connected to the another component, or an intermediate component may exist at the same time.

Unless otherwise defined, meanings of all technical and scientific terms used in this application are the same as those usually understood by a person skilled in the art to which this application belongs. In this application, terms used in the specification of this application are merely intended to describe objectives of the specific embodiments, but are not intended to limit this application.

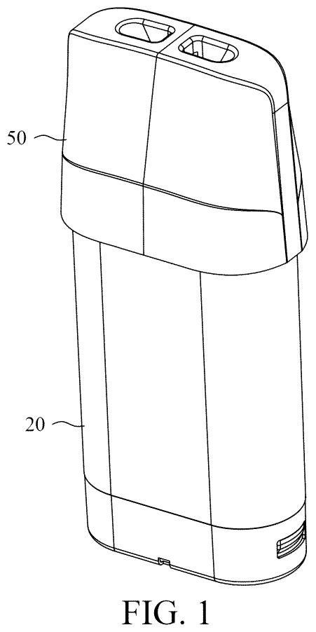

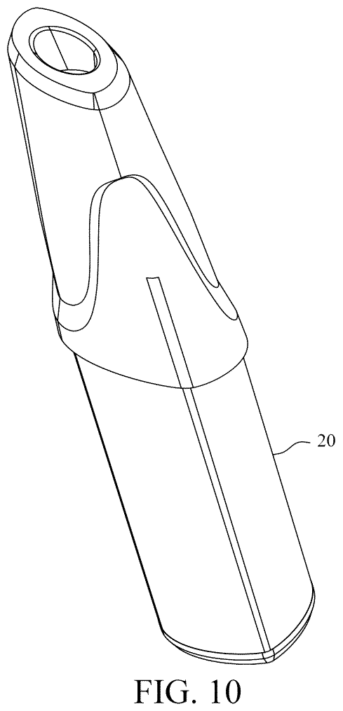

Embodiments of this application provide an electronic atomization device, and referring to FIG. 1 to FIG. 3 and referring to FIG. 10 to FIG. 12 in combination, the electronic atomization device includes an atomizer provided in any embodiment of this application.

The electronic atomization device is configured to atomize an aerosol-generation substrate to generate an aerosol for a user to inhale. The aerosol-generation substrate includes, but is not limited to, a drug, a nicotine-containing material, a nicotine-free material, and the like. The aerosol-generation substrate is also not limited to a liquid or a solid. In all the following embodiments, an example in which a liquid aerosol-generation substrate is used for description.

The electronic atomization device usually includes an atomizer and a power supply component. The power supply component is configured to supply power to the atomizer. The atomizer is detachably connected to the power supply component. Certainly, the electronic atomization device may further include a shell, and the atomizer and the power supply component may both be accommodated in the shell for case of use by the user. The atomizer converts electric energy into thermal energy. Under the action of the thermal energy, the aerosol-generation substrate is converted into an aerosol for the user to inhale. In this process, when an atomization core 30 of the atomizer sucks a liquid, a heating body of the atomization core 30 heats and atomizes the aerosol-generation substrate. The atomization core 30 continuously sucks the liquid during atomization, and external air enters a liquid storage cavity through a vent channel 80.

It is to be noted that, a specific type of the electronic atomization device provided in the embodiments of this application is not limited. For example, the electronic atomization device may be a medical atomization apparatus, or may be an air humidifier, or may be an e-cigarette or some other apparatuses that need to use an atomizer.

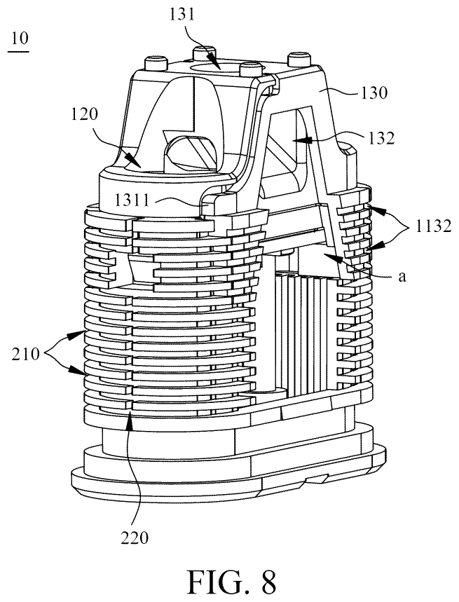

Embodiments of this application provide an atomizer. Referring to FIG. 1 to FIG. 17, the atomizer includes a housing 20, an atomization base 10, and a vent channel 80.

Referring to FIG. 2 and FIG. 11, the housing 20 is provided with an accommodating cavity c and an air outlet channel 21. An aerosol generated by the aerosol-generation substrate flows through the air outlet channel 21 for a user to inhale. It is to be noted that, a specific manner of using the atomizer is not limited herein. For example, the user may inhale the aerosol through the housing 20, or may inhale the aerosol by cooperating an additional suction nozzle 50 with the housing 20.

Referring to FIG. 2 and FIG. 11, at least a partial structure of the atomization base 10 is disposed in the accommodating cavity c. A liquid storage cavity b configured to store an aerosol-generation substrate is defined between the top wall 111 of the atomization base 10 and the housing 20. The atomization base 10 is formed with an atomization cavity a and at least one liquid flowing channel 120. The liquid flowing channel 120 is communicated between the liquid storage cavity b and the atomization cavity a. That is, the aerosol-generation substrate stored in the liquid storage cavity b may enter the atomization cavity a through the liquid flowing channel 120 for heating and atomization.

It is to be noted that, at least a partial structure of the atomization base 10 being disposed in the accommodating cavity c may be that a partial structure of the atomization base 10 is disposed in the accommodating cavity c, or may be that the entire structure of the atomization base 10 is disposed in the accommodating cavity c.

The aerosol-generation substrate in the liquid storage cavity b is guided into the atomization cavity a through the liquid flowing channel 120 for heating and atomization to generate an aerosol. After the aerosol-generation substrate in the liquid storage cavity b is consumed, external air enters the liquid storage cavity b through a vent outlet 81 of the vent channel 80 to balance a pressure in the liquid storage cavity b.

For a high-viscosity aerosol-generation substrate with dynamic viscosity exceeding 500 cp at a room temperature, the dynamic viscosity of the substrate decreases rapidly as the temperature rises. Because the substrate has poor fluidity at a low temperature, in the related art, the vent outlet 81 of the vent channel 80 is usually disposed near a heating body or near a liquid flowing opening of the liquid flowing channel 120. If the vent outlet 81 is disposed near the heating body, bubbles generated after ventilation still remain near the heating body and cannot be discharged. If the vent outlet is disposed near the liquid flowing opening of the liquid flowing channel 120, because the temperature near the liquid flowing opening is low, the viscosity of the aerosol-generation substrate is large. As a result, ventilation bubbles accumulate at and block the liquid flowing opening, and the liquid suction of the atomization core 30 is hindered accordingly. Unsmooth liquid flowing leads to dry heating of the atomization core 30, affecting the service life of the atomizer and the user experience of a user.

The atomizer provided in the embodiments of this application, the vent channel 80 is provided. The vent outlet 81 of the vent channel 80 is communicated with the liquid storage cavity b. The aerosol-generation substrate in the liquid storage cavity b is guided into the atomization cavity a through the liquid flowing channel 120 for heating and atomization to generate an aerosol. The aerosol passes through the air outlet channel 21 for a user to inhale. After the aerosol-generation substrate in the liquid storage cavity b is consumed, external air enters the liquid storage cavity b through the vent channel 80 to balance a pressure in the liquid storage cavity b. When flowing through the air outlet channel 21, the high-temperature aerosol generated from the heating and atomization exchanges heat with the side wall 112 of the air outlet channel 21. For example, the high-temperature aerosol performs convective heat exchange, condensing heat release, and the like. Therefore, the temperature of the side wall 112 of the air outlet channel 21 is increased, and the side wall 112 of the air outlet channel 21 conducts heat to the aerosol-generation substrate near the air outlet channel 21. The aerosol-generation substrate near the air outlet channel 21 absorbs heat, undergoes a temperature rise, and has reduced viscosity.

In this way, the vent outlet 81 is provided close to the air outlet channel 21, and bubbles generated at the vent outlet 81 form and spread in a region of the aerosol-generation substrate with low viscosity. That is, the vent outlet 81 is provided close to the air outlet channel 21. In one aspect, the aerosol-generation substrate near the vent outlet 81 may have low viscosity. This makes it easier for air to enter the liquid storage cavity b through the vent channel 80, so that while ventilation is improved, the vent channel 80 may be kept from being blocked. In another aspect, the aerosol-generation substrate near the air outlet channel 21 absorbs heat, undergoes a temperature rise, and has reduced viscosity. This makes it convenient for bubbles generated at the vent outlet 81 to spread in an extension direction of the air outlet channel 21, so that bubbles generated at the vent outlet 81 can be kept from spreading transversely and from blocking the liquid flowing channel 120. In this way, a case that bubbles get stuck in the liquid flowing channel 120 can be mitigated, thereby improving the service life of the atomizer and the user experience of a user.

The vent outlet 81 is disposed close to the air outlet channel 21. That is, the vent outlet 81 is disposed as near as possible to the air outlet channel 21. That is, the vent outlet 81 is located in a region in which the aerosol-generation substrate absorbs heat, undergoes a temperature rise, and has reduced viscosity.

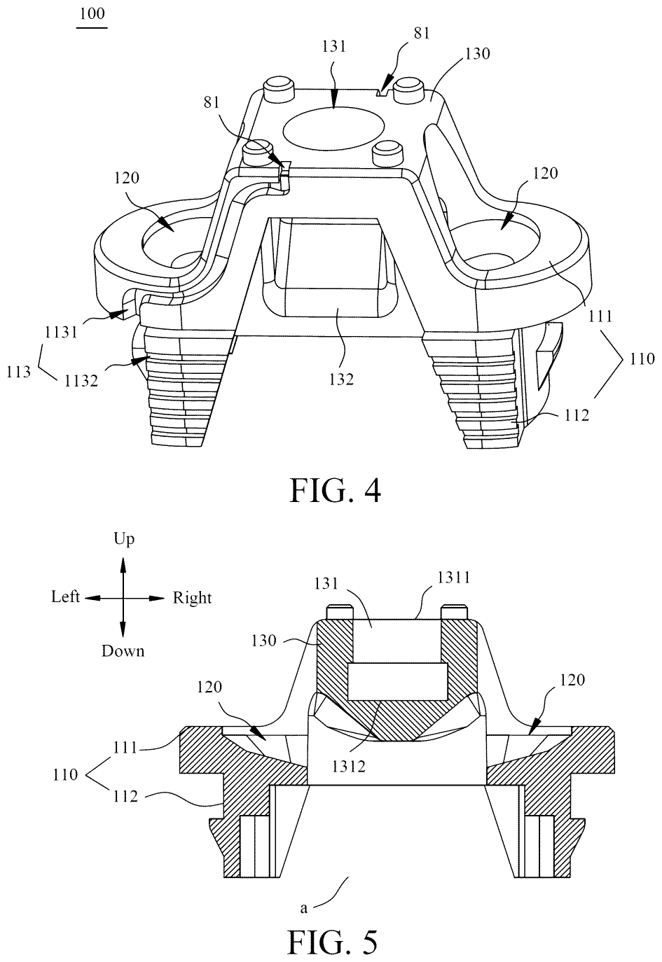

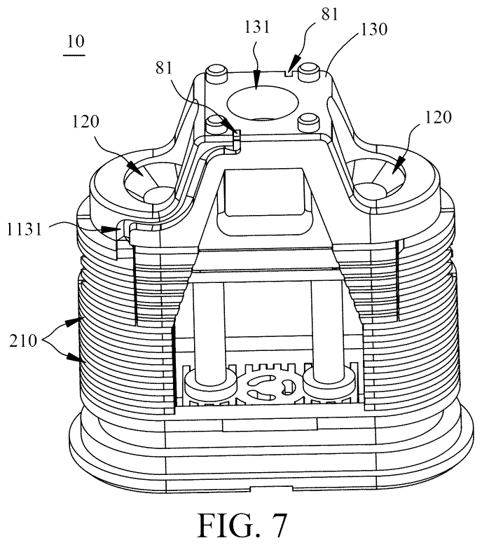

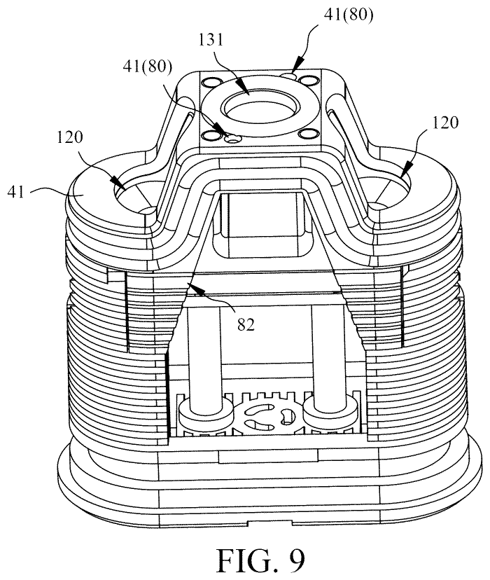

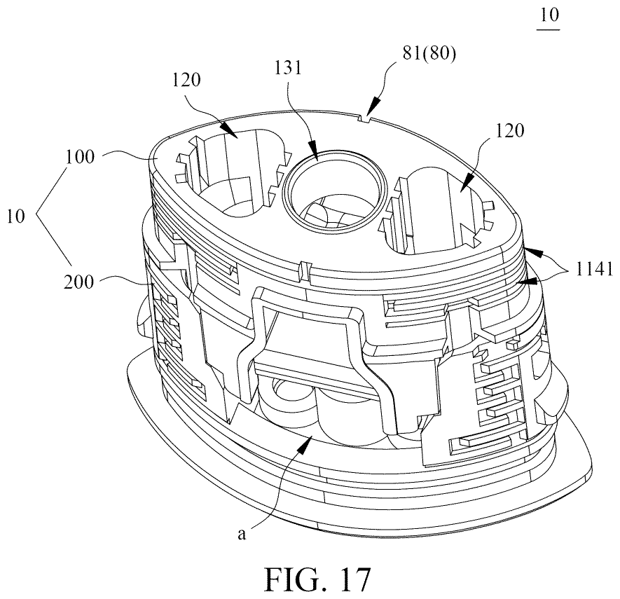

In an embodiment, a plurality of vent channels 80 are provided. For example, referring to FIG. 4, FIG. 7, and FIG. 9 and referring to FIG. 12 and FIG. 17 in combination, two vent channels 80 are provided. In this way, the plurality of vent channels 80 are provided to make it convenient for external air to enter the liquid storage cavity b through the vent channel 80 to improve ventilation efficiency, and a case that any vent channel 80 is blocked and as a result external air cannot enter the liquid storage cavity b can be avoided.

The vent channels 80 are symmetrically distributed along the central axis of the air outlet channel 21. In this way, air intake and air discharge between the vent channels 80 can be protected from interference, thereby further improving the ventilation efficiency.

In an embodiment, a plurality of liquid flowing channels 120 are provided. For example, referring to FIG. 4 and FIG. 5 and referring to FIG. 13 and FIG. 15 in combination, two liquid flowing channels 120 are provided. In this way, the plurality of liquid flowing channels 120 are provided to make it convenient for the aerosol-generation substrate in the liquid storage cavity b to be transferred to the atomization core 30 through the liquid flowing channel 120 for heating and atomization, to improve the atomization efficiency, and a case that any liquid flowing channel 120 is blocked, and the liquid suction of the atomization core 30 is hindered as a result, leading to the dry heating of the atomization core 30 can be further avoided.

The liquid flowing channels 120 are symmetrically distributed along the central axis of the air outlet channel 21. In this way, liquid flowing between the liquid flowing channels 120 can be protected from interference, so that the smoothness of liquid flowing can be improved.

In an embodiment, referring to FIG. 4 and FIG. 13, the vent outlet 81 is disposed away from the liquid inlet opening of the liquid flowing channel 120. That is, while the vent outlet 81 is disposed as close as possible to the air outlet channel 21, the vent outlet 81 is disposed as far as possible away from the liquid inlet opening of the liquid flowing channel 120. In this way, a possibility that bubbles generated at the vent outlet 81 block the liquid inlet opening of the liquid flowing channel 120 is further reduced.

For example, two liquid flowing channels 120 are symmetrically disposed on the atomization base 10. The vent outlet 81 is disposed in a direction perpendicular to the central axis of the two liquid flowing channels 120. In this way, the vent outlet 81 may be provided as far as possible away from the liquid inlet opening of the liquid flowing channel 120.

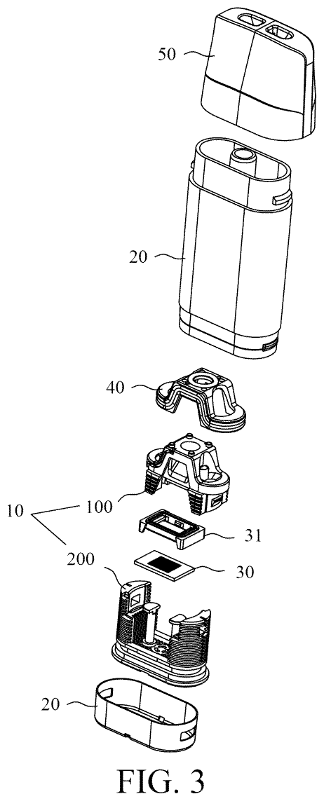

In an embodiment, referring to FIG. 2 and FIG. 3 and referring to FIG. 11 and FIG. 12 in combination, the atomizer includes the atomization core 30 disposed in the atomization cavity a. The atomization core 30 includes the heating body. The aerosol-generation substrate in the liquid storage cavity b is guided to the atomization core 30 through the liquid flowing channel 120. The heating body may perform heating and atomization on the aerosol-generation substrate. A seal gasket 31 may be disposed outside the atomization core 30, facilitating the mounting and the liquid suction of the atomization core 30.

In an embodiment, referring to FIG. 9 and FIG. 11, the vent channel 80 has a vent inlet 82 provided in the atomization cavity a. The vent inlet 82 is provided in a region in which the atomization base 10 is located at the periphery of the heating body, that is, is provided close to the heating body. That is, an airflow that enters the vent channel 80 through the vent inlet 82 is preheated by the heating body. That is, the airflow that enters the vent channel 80 is a preheated airflow and has a high temperature. In one aspect, the temperature of the aerosol-generation substrate at the vent outlet 81 can be kept from being reduced by a cold airflow that enters the liquid storage cavity b, to avoid affecting the smoothness of an airflow entering the liquid storage cavity b through the vent channel 80. In another aspect, the preheated airflow and the aerosol-generation substrate at the vent outlet 81 exchange heat, and the viscosity of the aerosol-generation substrate at the vent outlet 81 is further reduced, thereby further improving the ventilation efficiency.

In an embodiment, referring to FIG. 5 and FIG. 15, an air guide channel 131 and a vent opening 132 are opened in the atomization base 10. The air guide channel 131 includes an open end 1311 (that is, the upper end of the air guide channel 131 shown in FIG. 5 and FIG. 15, and the upper end has an opening) and a closed end 1312 (that is, the lower end of the air guide channel 131 shown in FIG. 5 and FIG. 15) opposite to the open end 1311. The vent opening 132 is located across the two sides of the central axis of the air guide channel 131 in a first direction (a front-back direction shown in FIG. 13). The air guide channel 131 is communicated with the atomization cavity a through the vent opening 132 and is communicated with the air outlet channel 21 through the open end 1311. The first direction is perpendicular to the central axis of the air guide channel 131. In this way, the aerosol in the atomization cavity a enters the air guide channel 131 through the vent opening 132, and then enters the air outlet channel 21 through the open end 1311 of the atomization cavity a, so that the space is effectively utilized, and the use of the user is facilitated.

In an implementation, the vent outlet 81 is provided in at least one side of the air guide channel 131 in the first direction (the front-back direction shown in FIG. 13). It may be understood that to effectively utilize a mounting space while avoiding interference between an airflow flow channel between the vent opening 132 and the air guide channel 131 and the liquid flowing channel 120, the vent opening 132 is located across the two sides of the central axis of the air guide channel 131 in the first direction. The liquid flowing channel 120 is located across the two sides of the central axis of the air guide channel 131 in a second direction (a left-right direction shown in FIG. 13) perpendicular to the first direction. The vent outlet 81 is disposed on at least one side of the air guide channel 131 in the first direction. That is, while the vent outlet 81 is disposed as close as possible to the air outlet channel 21, the vent outlet 81 is disposed as far as possible away from the liquid inlet opening of the liquid flowing channel 120. In this way, a possibility that bubbles generated at the vent outlet 81 block the liquid inlet opening of the liquid flowing channel 120 is further reduced.

Referring to FIG. 2 and FIG. 11, an air intake channel 22 is further formed inside the housing 20, the air outlet channel 21 is communicated with the top end of the atomization cavity a, and the air intake channel 22 is communicated with the bottom end of the atomization cavity a. That is, the air intake channel 22 is located in the bottom side of the atomization cavity a, and the air outlet channel 21 is located in the top side of the atomization cavity a. Optionally, one end of the air outlet channel 21 is communicated with the open end 1311 of the air guide channel 131 shown in some foregoing embodiments, and the other end of the air outlet channel 21 is communicated with the suction nozzle 50, to implement an air suction process.

In an embodiment, referring to FIG. 2 and FIG. 3 and referring to FIG. 11 and FIG. 12 in combination, the atomization base 10 includes a bottom atomization base 200 and a top atomization base 100, the atomization cavity a is defined between the bottom atomization base 200 and the top atomization base 100, and the air guide channel 131, the vent opening 132, and the liquid flowing channel 120 are opened in the top atomization base 100. The atomization core 30 is disposed in the atomization cavity a, and the liquid flowing channel 120 guides the aerosol-generation substrate to an atomization surface of the atomization core 30 located in the atomization cavity a. When the heating body in the atomizer is energized to convert electric energy into thermal energy, the liquid sucked by the atomization core 30 is atomized to form an aerosol, and the aerosol is discharged into the atomization cavity a. When an air suction action of an airflow is generated at the air outlet channel 21, the aerosol in the atomization cavity a enters the air outlet channel 21 for use by the user.

It is to be noted that, a specific structure of the atomizer is not limited herein. To better describe the structural form of the atomizer, two different embodiments are respectively described below. However, this application is not limited thereto.

An atomizer in a first embodiment is shown in FIG. 1 to FIG. 9:

In an embodiment, referring to FIG. 4 and FIG. 5, the top atomization base 100 includes a top base body 110 and a boss 130. The top base body 110 includes a top wall 111 and a side wall 112 surrounding the top wall 111. The top wall 111 and the side wall 112 surround to form an atomization cavity a that is independent of a liquid storage cavity b and provided with an opening at an end. The liquid flowing channels 120 are provided in the top wall 111. The air guide channel 131 is formed in the boss 130, and the vent outlet 81 is formed in the boss 130 and is provided close to the open end 1311. For example, FIG. 2 to FIG. 9 show a case in which two liquid flowing channels 120 are provided. One liquid flowing channel 120 is located on the left side of the top atomization base 100, and the other liquid flowing channel 120 is located on the right side of the top atomization base 100. Certainly, a plurality of liquid flowing channels 120 may be provided in each side. The liquid flowing channels may be provided according to an actual use. This is not specifically limited in the embodiments of this application.

It may be understood that, referring to FIG. 4 and FIG. 5, the boss 130 is disposed protruding from the top wall 111. The liquid flowing channel 120 is provided in the top wall 111. The vent outlet 81 is formed in the boss 130. That is, the vent outlet 81 is higher than the liquid flowing opening of the liquid flowing channel 120. In this way, bubbles generated at the vent outlet 81 spread upward, so that ventilation bubbles can be kept from blocking the liquid flowing opening of the liquid flowing channel 120. In this way, a case that bubbles get stuck in the liquid flowing channel 120 can be mitigated, thereby improving the service life of the atomizer and the user experience of a user. In addition, because the air guide channel 131 is communicated with the air outlet channel 21 through the open end 1311, in this way, the vent outlet 81 is provided close to the open end 1311, so that the vent outlet 81 can be provided as close as possible to the air outlet channel 21.

Referring to FIG. 4, the peripheral wall of the top base body 110 is formed with a first vent groove 113 communicated with the vent outlet 81, and the end of the first vent groove 113 away from the vent outlet 81 is communicated with the atomization cavity a. The aerosol-generation substrate flows into the liquid flowing channel 120 through the liquid storage cavity b. The liquid flowing channel 120 guides the aerosol-generation substrate to the atomization surface of the atomization core 30 located in the atomization cavity a. When the heating body in the atomizer is energized to convert electric energy into thermal energy, the liquid sucked by the atomization core 30 is atomized to form an aerosol, and the aerosol is discharged into the atomization cavity a. When an air suction action of an airflow is generated at the air outlet channel 21, the aerosol in the atomization cavity a enters the air outlet channel 21 for use by the user. Moreover, an airflow that enters from outside may be transferred to the vent outlet 81 through the first vent groove 113 and enters the liquid storage cavity b, to implement the ventilation in the liquid storage cavity b.

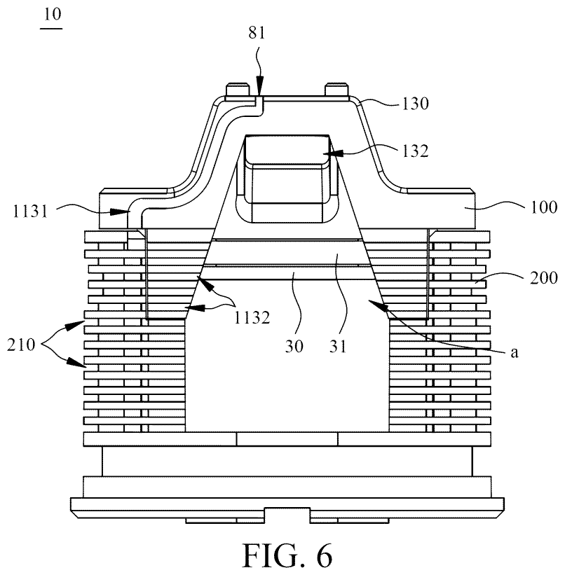

In an embodiment, referring to FIG. 4 and FIG. 6, the first vent groove 113 includes a first sub-groove 1131 communicated with the vent outlet 81 and a second sub-groove 1132 communicated with the atomization cavity a, the first sub-groove 1131 is provided in the top wall 111 and the peripheral wall of the boss 130, and the second sub-groove 1132 is provided in the peripheral wall of the side wall 112. In this embodiment, the end of the second sub-groove 1132 communicated with the atomization cavity a is disposed close to the heating body. An external airflow flows to the first sub-groove 1131 from the second sub-groove 1132, and then enters the liquid storage cavity b through the vent outlet 81 of the vent channel 80 to perform ventilation.

It is to be noted that, a specific arrangement position of the first sub-groove 1131 is not limited herein. For example, the first sub-groove 1131 is provided close to the air guide channel 131, the temperature near the air guide channel 131 is high, so that the viscosity of the aerosol-generation substrate that enters the first sub-groove 1131 can be reduced. This is conducive to the smoothness of an airflow entering the liquid storage cavity b through the vent channel 80.

It is to be noted that, the first sub-groove 1131 and the second sub-groove 1132 are communicated in various manners, and may be directly communicated, or may be indirectly communicated. For example, in an embodiment, referring to FIG. 6 to FIG. 9, the peripheral wall of the bottom atomization base 200 is formed with a second vent groove 210. The end of the first sub-groove 1131 away from the vent outlet 81 is communicated with the second sub-groove 1132 through the second vent groove 210. In this way, the external airflow flows into the second sub-groove 1132 from the atomizer, then flows through the second vent groove 210 from the second sub-groove 1132, flows into the first sub-groove 1131, and then enters the liquid storage cavity b through the vent outlet 81 of the vent channel 80 to perform ventilation.

In some other embodiments, the end of the first sub-groove 1131 away from the vent outlet 81 is communicated with the second sub-groove 1132. That is, the first sub-groove 1131 is directly communicated with the second sub-groove 1132.

In an embodiment, referring to FIG. 6 to FIG. 9, a plurality of second vent grooves 210 are provided, and the second vent grooves 210 are sequentially communicated. The plurality of sequentially communicated second vent grooves 210 are configured to keep the aerosol-generation substrate in the liquid storage cavity b from entering the vent channel 80 through the vent outlet 81, thereby avoiding liquid leakage. For example, when the air pressure in the liquid storage cavity b decreases (for example, when the electronic atomization device is transported by a plane), the volumes of the bubbles in the liquid storage cavity b increase. The aerosol-generation substrate that overflows through the second sub-groove 1132 is accommodated in the plurality of sequentially communicated second vent grooves 210, thereby mitigating liquid leakage. When the air pressure in the liquid storage cavity b becomes normal again, the aerosol-generation substrate stored in the second vent groove 210 may flow back into the liquid storage cavity b through the second sub-groove 1132, thereby mitigating negative-pressure liquid leakage. In an embodiment, continuing to refer to FIG. 6 to FIG. 9, the peripheral wall of the bottom atomization base 200 is formed with a first vent groove 220 communicating the second vent grooves 210. That is, the second vent grooves 210 are communicated through the first vent groove 220. One or more first vent grooves 220 may be provided, and the quantity is not limited herein.

An arrangement manner of the plurality of second vent grooves 210 is not limited herein. For example, continuing to refer to FIG. 6 to FIG. 9, the second vent grooves 210 are provided at intervals in the height direction of the bottom atomization base 200, and the second vent grooves 210 are provided in parallel.

In an embodiment, continuing to refer to FIG. 6 to FIG. 9, a plurality of second sub-grooves 1132 are provided. The second sub-grooves 1132 are joined to and communicated with the corresponding second vent grooves 210. It may be understood that, the plurality of second sub-grooves 1132 are communicated with the atomization cavity a. That is, the airflow in the atomization cavity a may enter the second vent grooves 210 through the plurality of second sub-grooves 1132 simultaneously, and enter the liquid storage cavity b through the second vent grooves 210. In this way, the plurality of second sub-grooves 1132 make it convenient for more of the airflow in the atomization cavity a to enter the liquid storage cavity b, thereby improving the ventilation efficiency, and can further avoid a case in which any one of the second sub-grooves 1132 is blocked and ventilation fails.

In an embodiment, referring to FIG. 2, FIG. 3, and FIG. 9, the atomizer further includes a first seal member 40. The first seal member 40 is disposed at the top end of the top atomization base 100. Some mounting gaps exist between the top atomization base 100 and the side wall 112 of the accommodating cavity c. The first seal member 40 is disposed at the top end of the top atomization base 100, and is configured to seal the mounting gaps between the top atomization base 100 and the side wall 112 of the accommodating cavity c, so that the aerosol-generation substrate in the liquid storage cavity b can be kept from flowing through the mounting gaps between the top atomization base 100 and the side wall 112 of the accommodating cavity c, thereby avoiding liquid leakage.

In an embodiment, one part of the vent channel 80 is defined between the first seal member 40 and the groove wall of the first sub-groove 1131, and the other part of the vent channel 80 is defined between the side wall 112 of the accommodating cavity c and the groove walls of the second sub-groove 1132 and the second vent groove. A first vent hole 41 is further opened in the first seal member 40, and the first vent hole 41 is communicated between the liquid storage cavity b and the vent outlet 81. In this way, the external airflow flows to the vent outlet 81 through the vent channel 80, and then enters the liquid storage cavity b through the vent hole, to implement the ventilation of the liquid storage cavity b.

An atomizer in a second embodiment is shown in FIG. 10 to FIG. 17:

In an embodiment, referring to FIG. 13 to FIG. 15, a liquid inlet opening of the liquid flowing channel 120, the open end 1311, and the vent outlet 81 are all formed at the top end of the top atomization base 100. The liquid storage cavity b is defined between the top end of the top atomization base 100 and the housing 20. For example, the open end 1311 is disposed at a middle position of the top end of the top atomization base 100. The liquid inlet openings of two liquid flowing channels 120 are symmetrically distributed along the central axis of the air outlet channel 21. Two vent outlets 81 are symmetrically distributed along the central axis of the air outlet channel 21.

In an embodiment, continuing to refer to FIG. 13 to FIG. 15, the top atomization base 100 is formed with a third vent groove 114 communicated with the vent outlet 81. The end of the third vent groove 114 away from the vent outlet 81 is communicated with the atomization cavity a. An airflow that enters from outside may be transferred to the vent outlet 81 through the third vent groove 114 and enters the liquid storage cavity b, to implement the ventilation in the liquid storage cavity b.

In an embodiment, referring to FIG. 16 and FIG. 17, the bottom atomization base 200 is formed with a fourth vent groove 230 communicated with the atomization cavity a, and the end of the third vent groove 114 away from the vent outlet 81 is communicated with the atomization cavity a through the fourth vent groove 230. In this embodiment, the end of the fourth vent groove 230 communicated with the atomization cavity a is disposed close to the heating body. An external airflow flows to the third vent groove 114 from the fourth vent groove 230, and then enters the liquid storage cavity b through the vent outlet 81 of the vent channel 80 to perform ventilation.

In an embodiment, referring to FIG. 13 and FIG. 14, the third vent groove 114 includes a plurality of third sub-grooves 1141 formed in the peripheral wall of the top atomization base 100. The third sub-grooves 1141 are sequentially communicated. The plurality of sequentially communicated third sub-grooves 1141 are configured to keep the aerosol-generation substrate in the liquid storage cavity b from entering the vent channel 80 through the vent outlet 81, thereby avoiding liquid leakage. For example, when the air pressure in the liquid storage cavity b decreases (for example, when the electronic atomization device is transported by a plane), the volumes of the bubbles in the liquid storage cavity b increase. The aerosol-generation substrate that overflows through the vent outlet 81 is accommodated in the plurality of sequentially communicated third sub-grooves 1141, thereby mitigating liquid leakage. When the air pressure in the liquid storage cavity b becomes normal again, the aerosol-generation substrate stored in the third sub-grooves 1141 may flow back into the liquid storage cavity b through the vent outlet 81, thereby mitigating negative-pressure liquid leakage.

In an embodiment, the peripheral wall of the top atomization base 100 is formed with a second vent groove communicating the third sub-grooves 1141. That is, the third sub-grooves 1141 are communicated through the second vent groove. One or more second vent grooves may be provided, and the quantity is not limited herein.

The fourth vent groove 230 is communicated with the first third sub-groove 1141 in an airflow flowing direction, and the vent outlet 81 is communicated with the last third sub-groove 1141 in the airflow flowing direction. In this way, the external airflow flows to the first third sub-groove 1141 in the airflow flowing direction from the fourth vent groove 230, flows to the vent outlet 81 through the last third sub-groove 1141 in the airflow flowing direction, and then enters the liquid storage cavity b through the vent outlet 81 to perform ventilation.

In an embodiment, referring to FIG. 16, the bottom atomization base 200 includes a body 240 and a connection portion 250 disposed protruding from the body 240, the bottom portion of the top atomization base 100 abuts against the body 240, and the peripheral wall of the top atomization base 100 is clamped at the connection portion 250. During assembly, the top atomization base 100 is placed near the body 240. When the bottom portion of the top atomization base 100 abuts against the body 240, the peripheral wall of the top atomization base 100 is clamped to the connection portion 250, to implement the connection between the top atomization base 100 and the bottom atomization base 200.

A specific manner of clamping the top atomization base 100 to the bottom atomization base 200 is not limited herein. For example, in an embodiment, the peripheral wall of the top atomization base 100 is provided with a fastener, and the side wall 112 of the connection portion 250 is provided with a clamping hole. When the bottom portion of the top atomization base 100 abuts against the body 240, the fastener and the clamping hole are clamped in cooperation, to implement the connection between the top atomization base 100 and the bottom atomization base 200.

In an embodiment, referring to FIG. 16, the side wall 112 of the atomization cavity a forms one part of the fourth vent groove 230, the top end of the body 240 forms the other part of the fourth vent groove 230, the third vent groove 114 includes a fourth sub-groove 1142 extending inward along the peripheral wall of the top atomization base 100, and the fourth vent groove 230 is communicated with the first third sub-groove 1141 in the airflow flowing direction through the fourth sub-groove 1142. That is, one part of the fourth vent groove 230 is formed in the side wall 112 of the atomization cavity a, and is communicated with the atomization cavity a. The other part of the fourth vent groove 230 is formed at the top end of the body 240, and is configured to be communicated with the third vent groove 114. The third vent groove 114 includes the fourth sub-groove 1142 extending inward along the peripheral wall of the top atomization base 100. That is, the fourth sub-groove 1142 is joined to and communicated with the fourth vent groove 230 formed at the top end of the body 240. In this way, the fourth vent groove 230 is communicated with the first third sub-groove 1141 in the airflow flowing direction through the fourth sub-groove 1142.

In an embodiment, referring to FIG. 11 and FIG. 12, the atomizer further includes a second seal member 90. The second seal member 90 is disposed at the top end of the top atomization base 100. Some mounting gaps exist between the top atomization base 100 and the side wall 112 of the accommodating cavity c. The second seal member 90 is disposed at the top end of the top atomization base 100, and is configured to seal the mounting gaps between the top atomization base 100 and the side wall 112 of the accommodating cavity c, so that the aerosol-generation substrate in the liquid storage cavity b can be kept from flowing through the mounting gaps between the top atomization base 100 and the side wall 112 of the accommodating cavity c, thereby avoiding liquid leakage.

One part of the vent channel 80 is defined between the second seal member 90 and the groove walls of the third sub-grooves 1141, and the other part of the vent channel 80 is defined between the top atomization base 100 and the bottom atomization base 200. A second vent hole 91 is further opened in the second seal member 90, and the second vent hole 91 is communicated between the liquid storage cavity b and the vent outlet 81. In this way, the external airflow flows to the vent outlet 81 through the vent channel 80, and then enters the liquid storage cavity b through the vent hole, to implement the ventilation of the liquid storage cavity b.

An electronic atomization device usually includes an atomizer and a power supply component. The power supply component is configured to supply power to the atomizer. The atomizer converts electric energy into thermal energy. Under the action of the thermal energy, an aerosol-generation substrate is converted into an aerosol for a user to inhale. In this process, after the atomization core sucks a liquid, a heating element heats and atomizes the aerosol-generation substrate. Because gaps exist in the atomization core, the atomization core continuously sucks the liquid during atomization. External air enters the liquid storage cavity through the gaps (or other gaps, for example, other vent structures formed by some structures and a liquid storage shell form, and the like) in the atomization core.

The inventor of this application has noticed that in a process in which external air enters the liquid storage cavity through an atomization core, because the aerosol-generation substrate has excessively high viscosity, bubbles with different sizes are formed when the air enters the liquid storage. When there are too many bubbles or excessively large bubbles, bubbles tend to accumulate above a liquid suction surface of the atomization core, bubbles get stuck in a liquid flowing channel communicated between the top atomization base and the liquid storage cavity, and as a result the liquid suction of the atomization core is hindered. The atomization core performs dry heating due to unsmooth liquid flowing, affecting the use of the user and the service life of the atomizer.

Based on this, in the embodiments of this application, the structure of the liquid flowing channel of the top atomization base is changed to mitigate a case that bubbles get stuck in the liquid flowing channel. The related description of the top atomization base provided in the embodiments of this application is provided below with reference to the related description of some embodiments.

It is to be noted that, the top atomization base disclosed in the embodiments of this application may be used in a medical atomization apparatus, or may be used in an air humidifier, or may further be used in an e-cigarette or some other apparatuses that need to use an atomizer. This is not specifically limited in the embodiments of this application. The structure of the top atomization base in some embodiments is used as an example for description below. However, this application is not limited thereto.

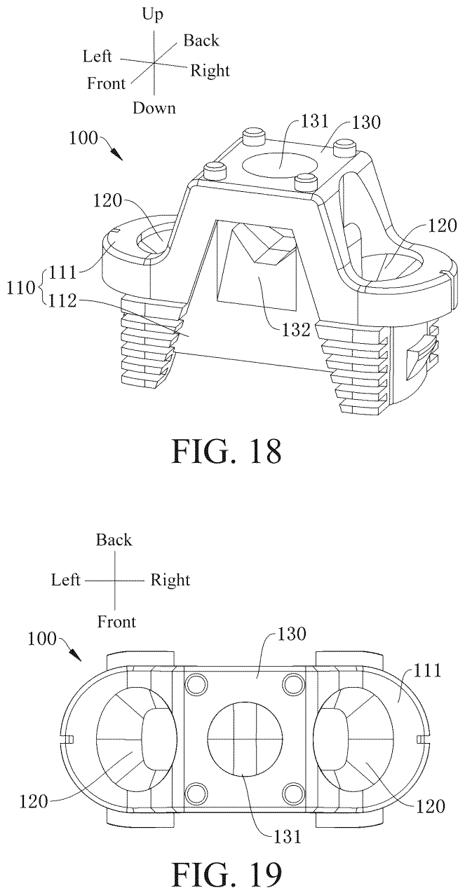

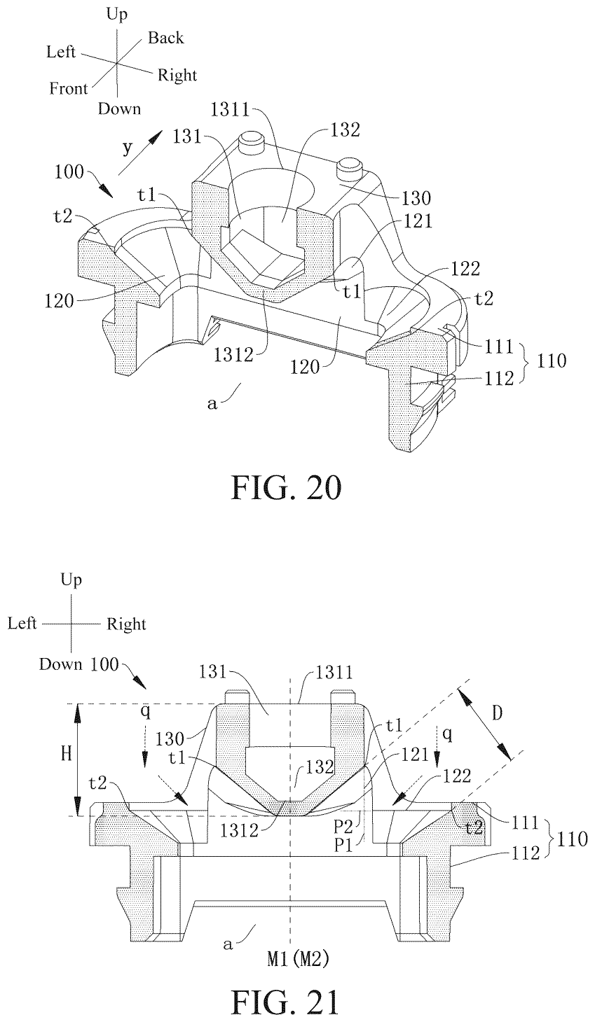

FIG. 18 is a schematic structural diagram of a top atomization base 100 in an implementation of an embodiment of this application from a viewing angle. FIG. 19 is a schematic structural diagram of a top atomization base 100 in an implementation of an embodiment of this application from another viewing angle. FIG. 20 is a schematic structural cross-sectional view of a top atomization base 100 in an implementation of an embodiment of this application from a viewing angle. FIG. 21 is a schematic structural cross-sectional view of a top atomization base 100 in an implementation of an embodiment of this application from another viewing angle. For case of description, only parts related to the embodiments of this application are shown.

For case of understanding, as shown in FIG. 18, the above in the drawing is defined as the above, the below in the drawing is defined as the below, the inside of the left in the drawing is defined as the left, the outside of the right of the drawing is defined as the right, the outside of the left of the drawing is defined as the front side, and the inside of the right in the drawing is defined as the rear side. The directions defined relative to the structure provided in the embodiments in FIG. 18 are still used in subsequent drawings. It is to be noted that, the foregoing definitions are only used for description, but cannot understood as a limitation to this application. It may be understood that FIG. 19 is a top view of FIG. 18, FIG. 20 is a three-dimensional cross-sectional view of FIG. 18, and FIG. 21 is a schematic cross-sectional front view of FIG. 18.

Referring to FIG. 18 and FIG. 19, embodiments of this application provide a top atomization base 100. The top atomization base 100 is communicated with a liquid storage cavity b of an atomizer. The liquid storage cavity b is configured to store an aerosol-generation substrate. The aerosol-generation substrate may be oil or another liquid. The top atomization base 100 includes a top base body 110 and at least two liquid flowing channels 120. The top base body 110 includes a top wall 111 and a side wall 112 surrounding the top wall 111, the top wall 111 and the side wall 112 surround to form an atomization cavity a that is independent of the liquid storage cavity b and provided with an opening at an end. All the liquid flowing channels 120 are provided in the top wall 111, and each liquid flowing channel 120 is communicated between the liquid storage cavity b and the atomization cavity a. For example, FIG. 18 to FIG. 21 show a case in which two liquid flowing channels 120 are provided. One liquid flowing channel 120 is located on the left side, and the other liquid flowing channel 120 is located on the right side of the top atomization base 100. Certainly, a plurality of liquid flowing channels 120 may be provided in each side. The liquid flowing channels may be provided according to an actual use. This is not specifically limited in the embodiments of this application.

Referring to FIG. 20 and FIG. 21 and referring to FIG. 18 and FIG. 19 in combination, the central axis of the atomization cavity a is defined as a first central axis MI. A first corner t1 disposed close to the first central axis M1 and a second corner t2 disposed away from the central axis of the atomization cavity a are provided inside each liquid flowing channel 120. The first corner t1 is located upstream the second corner t2 in a liquid inlet direction q (that is, a direction of entering the atomization cavity a from top to bottom shown in the figure). If the first corner t1 and the second corner t2 are located at a same horizontal line, the outlet of the liquid flowing channel 120 becomes small, and the aerosol-generation substrate is prone to blockage at a turning, causing unsmooth liquid flowing. FIG. 21 is used as an example. In the liquid inlet direction q, because the first corner t1 is located upstream the second corner t2, that is, the first corner t1 and the second corner t2 have a difference of a height H in a vertical direction, the aerosol-generation substrate can be scattered at the first corner t1 and the second corner t2, thereby expanding a liquid inlet channel, to avoid blockage due to simultaneous turning. In this way, a flow rate of the aerosol-generation substrate can be increased, and an escape space for bubbles generated when air passes through the outlet of the liquid flowing channel 120 is enlarged, to mitigate unsmooth liquid flowing, thereby improving the service life of the atomizer, and improving the user experience of a user.

It is to be noted that, the outlet of the liquid flowing channel 120 may tilt toward the first central axis M1, or may tilt away from the first central axis M1. That is, the first corner t1 and the second corner t2 may enable the inner wall of the liquid flowing channel 120 to turn toward the first central axis M1, or the first corner t1 and the second corner t2 may enable the inner wall of the liquid flowing channel 120 to turn away from the first central axis M1. Certainly, the turning directions of the first corner t1 and the second corner t2 may further be different. For example, as shown in FIG. 21, FIG. 21 shows a case in which the first corner t1 and the second corner t2 have the same turning direction, and both turn toward a first central axis. The turning directions may be selected according to an actual use. This is not specifically limited in the embodiments of this application.

In some embodiments, continuing to refer to FIG. 20 and FIG. 21, each liquid flowing channel 120 includes a first liquid inlet opening 121 provided close to the central axis of the atomization cavity a and a second liquid inlet opening 122 provided away from the central axis of the atomization cavity a. The first liquid inlet opening 121 and the second liquid inlet opening 122 are communicated with each other. A plane in which the first liquid inlet opening 121 is located is defined as a first plane P1, and a plane in which the second liquid inlet opening 122 is located is defined as a second plane P2. In the liquid inlet direction q, the first plane P1 is located above the second plane P2. That is, in the vertical direction before liquid intake, the first plane P1 is located above the second plane P2, and in the vertical direction after liquid intake, the first plane P1 is located below the second plane P2. The first corner t1 is located on a side of the first liquid inlet opening 121, and the second corner t2 is located on a side of the second liquid inlet opening 122.

Because the first liquid inlet opening 121 and the second liquid inlet opening 122 are located in different planes, compared with that the liquid inlet openings are liquid inlet openings in one plane, the liquid inlet openings are expanded, and a liquid flowing process before the aerosol-generation substrate reaches the corners smoother.

In some embodiments, continuing to refer to FIG. 20 and FIG. 21 and referring to FIG. 18 and FIG. 19 in combination, the top atomization base 100 further includes a boss 130 (a case in which the boss 130 extends upward is shown in the figure) disposed protruding from the top wall 111. At least a part of the first liquid inlet opening 121 in each liquid flowing channel 120 is opened in the boss 130, and the second liquid inlet opening 122 is opened in the top wall 111. In this way, because the boss 130 is provided, the liquid inlet openings can be enlarged, and the weight can further be reduced. Certainly, in some other embodiments, the boss 130 may not be disposed, and the first liquid inlet opening 121 and the second liquid inlet opening 122 located in different planes are only provided in the top wall 111 to improve liquid intake. This may be selected according to an actual use. This is not specifically limited in the embodiments of this application.

In some embodiments, continuing to refer to FIG. 20 and FIG. 21 and referring to FIG. 18 and FIG. 19 in combination, an air guide channel 131 is opened in the boss 130. All the liquid flowing channels 120 are distributed at the periphery of the air guide channel 131 in a circumferential direction. The first corner t1 in each liquid flowing channel 120 is located on the inner side close to the air guide channel 131, and the second corner t2 is located on the outer side away from the air guide channel 131. For example, FIG. 18 to FIG. 21 show a case in which two liquid flowing channels 120 are provided and the two liquid flowing channels 120 are respectively located on the left side and the right side of the air guide channel 131. Certainly, another quantity of liquid flowing channels 120 may be disposed surrounding the air guide channel 131. This may be selected according to an actual use. This is not specifically limited in the embodiments of this application. In this way, the space can be effectively utilized.

In some embodiments, continuing to refer to FIG. 20 and FIG. 21, the air guide channel 131 includes an open end 1311 (that is, the upper end of the air guide channel 131 shown in FIG. 20 and FIG. 21, and the upper end has an opening) and a closed end 1312 (that is, the lower end of the air guide channel 131 shown in FIG. 20 and FIG. 21) opposite to the open end 1311. The central axis of the air guide channel 131 is defined as the second central axis M2. The boss 130 is further provided with a vent opening 132 located across two sides of the second central axis M2 in a first direction y (the front-back direction shown in FIG. 20 and FIG. 21). The vent opening 132 is configured to communicate the air guide channel 131 and the vent opening 132 of the atomization cavity a. The first direction y is perpendicular to the central axis of the air guide channel 131. In this way, the aerosol in the atomization cavity a enters the air guide channel 131 through the vent opening 132, so that the space is effectively utilized, and the use of the user is facilitated. Specifically, in some embodiments, continuing to refer to FIG. 21, the planes in which the closed end 1312 of the air guide channel 131 and the second liquid inlet opening 122 are located are coplanar or in parallel. In this way, the length of the air guide channel 131 may be set according to an actual use.

In some embodiments, continuing to refer to FIG. 21 and referring to FIG. 9 discussed below in combination, an atomization core 30 of the atomizer is accommodated in the atomization cavity a. In the direction of the second central axis M2 (that is, the vertical direction shown in FIG. 21), a minimum distance L between the surface of the closed end 1312 of the air guide channel 131 facing a side of the atomization cavity a and the top portion of the atomization core 30 ranges from 1 mm to 3 mm. In this way, the distance L between the closed end 1312 of the air guide channel 131 and the top portion of the atomization core 30 is designed, and the escape space of bubbles and the space of the atomization cavity a located at the top portion of the atomization core 30 are further enlarged, thereby mitigating the problem of unsmooth liquid flowing.

In some embodiments, continuing to refer to FIG. 21, the central axis of the air guide channel 131 and the central axis of the atomization cavity a are parallel to each other and/or coincide. That is, the first central axis M1 and the second central axis M2 are parallel to each other and/or coincide. FIG. 21 is used as an example to show a case in which the first central axis M1 and the second central axis M2 coincide. In this way, it is convenient for the aerosol in the atomization cavity a to enter the air guide channel 131, and in addition, the use of the user is facilitated.

In some embodiments, continuing to refer to FIG. 21, in the direction of the central axis of the air guide channel 131, that is, the direction of the second central axis M2, the height H of the boss 130 ranges from 1.8 mm to 5.5 mm. In this way, the liquid inlet opening of the liquid flowing channel 120 can be expanded to facilitate liquid flowing, and the space of the air guide channel 131 can be further increased to facilitate air guidance.

In some embodiments, continuing to refer to FIG. 21, in the liquid inlet direction q, a cross-sectional diameter D of a part of each liquid flowing channel 120 located downstream the first corner t1 of the liquid flowing channel ranges from 2 mm to 4 mm. In this way, the corresponding cross-sectional diameter D may be designed to obtain the required escape space for expanded bubbles.

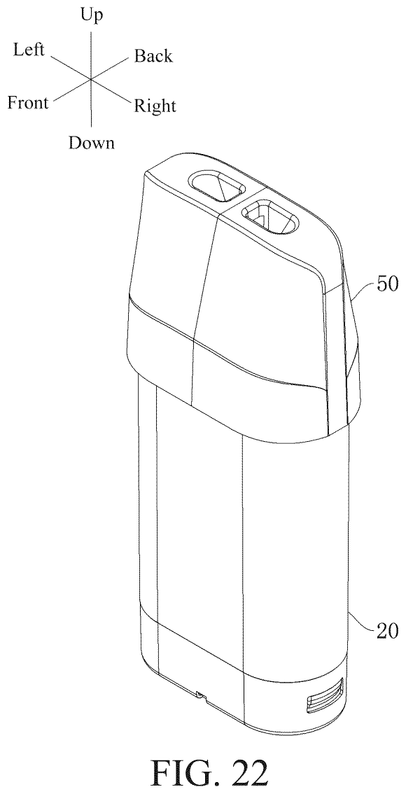

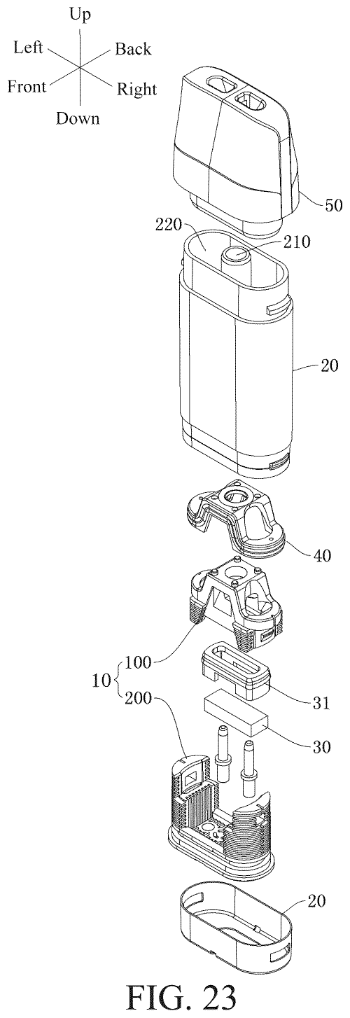

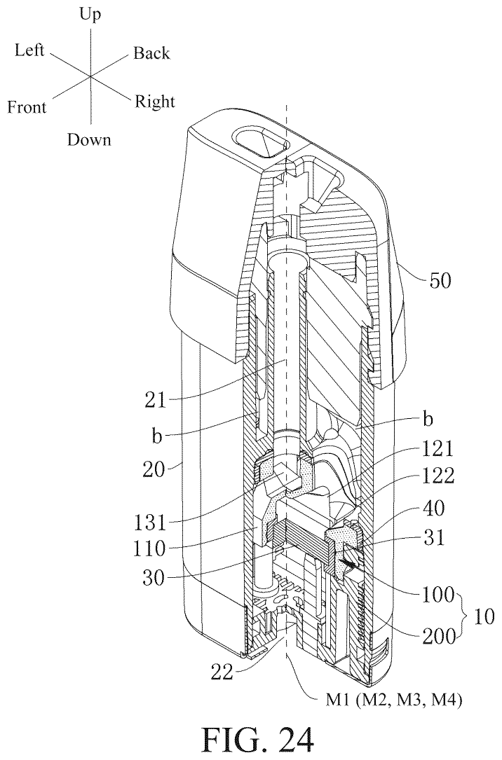

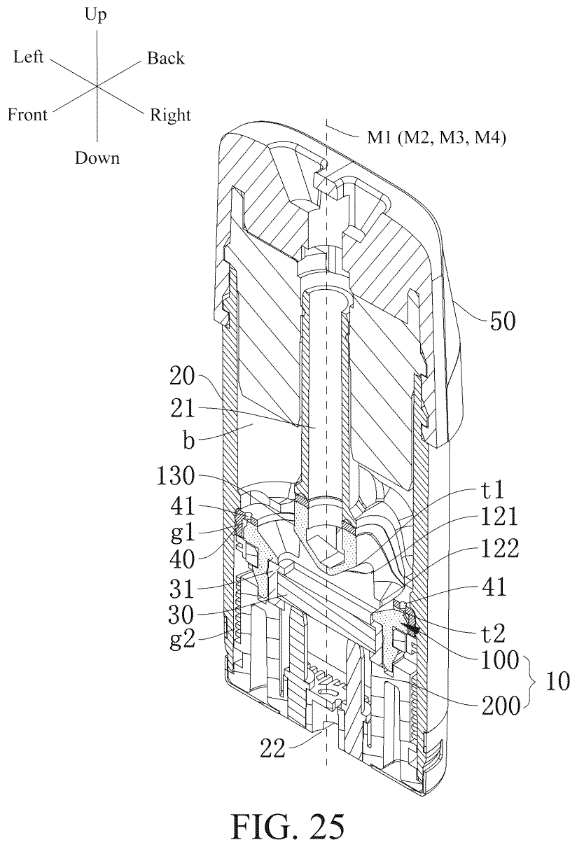

FIG. 22 is a schematic structural diagram of an atomizer in an implementation of an embodiment of this application. FIG. 23 is a schematic structural exploded view of an atomizer in an implementation of an embodiment of this application. FIG. 24 is a schematic structural cross-sectional view of an atomizer in an implementation of an embodiment of this application from a viewing angle. FIG. 25 is a schematic structural cross-sectional view of an atomizer in an implementation of an embodiment of this application from another viewing angle. For case of description, only parts related to the embodiments of this application are shown.

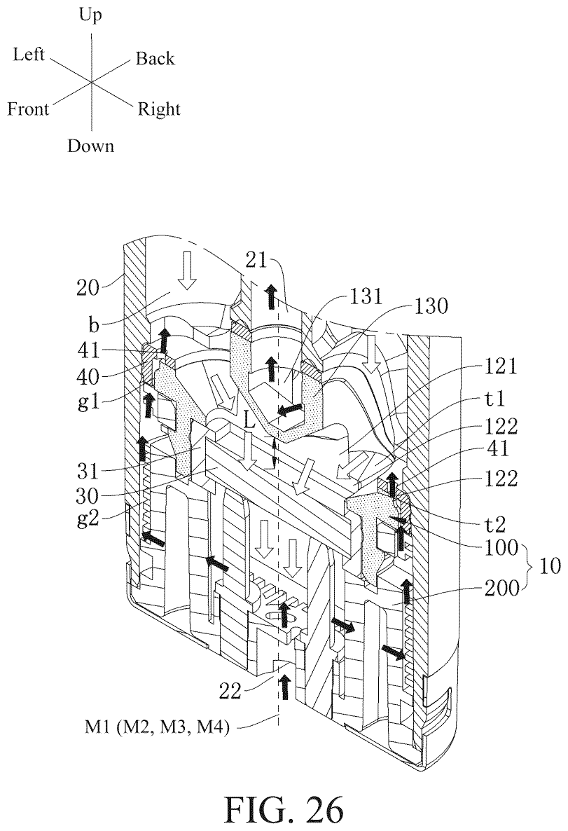

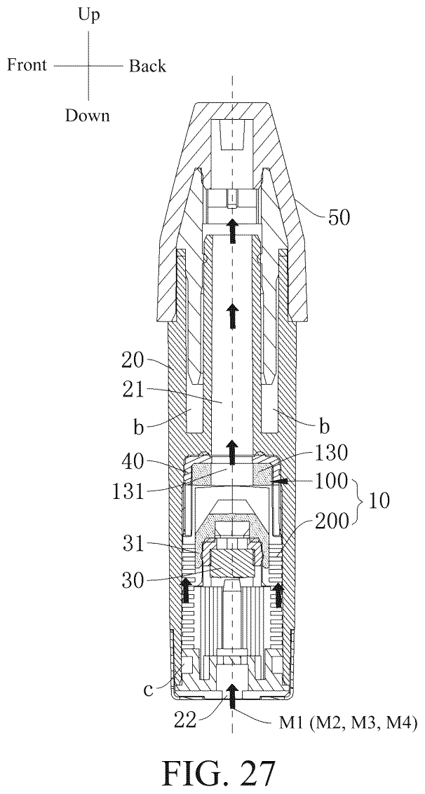

Based on the same inventive concept, as shown in FIG. 22 to FIG. 25, embodiments of this application provide an atomizer. The atomizer includes a housing 20, an atomization base 10, and an atomization core 30. With reference to FIG. 26 and FIG. 27 shown below, the housing 20 is provided with a liquid storage cavity b and an accommodating cavity c that are independent each other. In this way, different working processes can be implemented, to facilitate liquid flowing. The atomization base 10 is disposed in the accommodating cavity c, the atomization base 10 includes a bottom atomization base 200 and the top atomization base 100 in some foregoing embodiments, and the bottom atomization base 200 is provided with an opening to cooperate with the top atomization base 100 to form an atomization cavity a. The atomization core 30 is disposed in the atomization cavity a, and a seal gasket 31 may be disposed outside the atomization core 30, facilitating the mounting and the liquid suction of the atomization core 30. The liquid flowing channel 120 is configured to guide an aerosol-generation substrate in the liquid storage cavity b to the atomization core 30. In this way, because the top atomization base 100 in the foregoing some embodiments is used, the liquid flowing in the atomizer is smooth. The liquid suction process of the atomization core 30 is improved, to avoid the dry heating of the atomization core 30, thereby improving the service life of the atomizer and the user experience of a user.

In some embodiments, continuing to refer to FIG. 24 and FIG. 25, an air outlet channel 21 and an air intake channel 22 are further formed inside the housing 20, the air outlet channel 21 is communicated with the top end of the atomization cavity a, and the air intake channel 22 is communicated with the bottom end of the atomization cavity a. That is, the air intake channel 22 is located in the bottom side of the atomization cavity a, and the air outlet channel 21 is located in the top side of the atomization cavity a. Optionally, one end of the air outlet channel 21 is communicated with the open end 1311 of the air guide channel 131 shown in some foregoing embodiments, and the other end of the air outlet channel 21 is communicated with the suction nozzle 50, to implement an air suction process. The central axis of the air intake channel 22 is defined as a third central axis M3, and the central axis of the air outlet channel 21 is defined as a fourth central axis M4. The third central axis M3 and the fourth central axis M4 are parallel to each other and/or coincide. Specifically, the third central axis M3 and the fourth central axis M4 may both be vertically disposed or may be disposed tilting at an angle. For example, FIG. 24 and FIG. 25 show a case in which every two of the first central axis M1, the second central axis M2, the third central axis M3, and the fourth central axis M4 are vertically disposed and coincide. This may be set according to an actual use. This is not specifically limited in the embodiments of this application.

FIG. 26 is a partial schematic structural enlarged view of FIG. 25. FIG. 27 is a schematic structural cross-sectional view of an atomizer in an implementation of an embodiment of this application from still another viewing angle. For case of description, only parts related to the embodiments of this application are shown.

Referring to FIG. 26 and FIG. 27 and referring to FIG. 25 in combination, the atomizer further includes a seal member 40. The seal member 40 is disposed at the top end of the top atomization base 100. A first gap channel g1 is formed between the seal member 40 and the top atomization base 100, a second gap channel g2 communicated with the air intake channel 22 is formed between the atomization base 10 and the accommodating cavity c, and the first gap channel g1 is communicated with the air intake channel 22 through the second gap channel g2. A vent hole 41 is further opened in the seal member 40, and the vent hole 41 is communicated between the liquid storage cavity b and the first gap channel g1, to communicate the liquid storage cavity b with the air intake channel 22. FIG. 26 and FIG. 27 are used as an example. The black-frame arrows show a process of liquid flowing, and the black arrows show an air suction process and an air intake process in the figures. The aerosol-generation substrate flows into the liquid flowing channel 120 through the liquid storage cavity b. The liquid flowing channel 120 guides the aerosol-generation substrate to the atomization surface of the atomization core 30 located in the atomization cavity a. When the heating body (not labeled in the figure) in the atomizer is energized to convert electric energy into thermal energy, the liquid sucked by the atomization core 30 is atomized to form an aerosol, and the aerosol is discharged into the atomization cavity a. When an air suction action of an airflow is generated at the suction nozzle 50, the aerosol in the atomization cavity a enters the air outlet channel 21 and reaches the suction nozzle 50 for use by the user. Moreover, another part of a gas that enters the air intake channel 22 may sequentially pass through the second gap channel g2, the first gap channel g1, and the vent hole 41 to enter the liquid storage cavity b. In this way, the ventilation of the liquid storage cavity b can be improved through the vent hole 41, thereby further mitigating the case that bubbles get stuck.

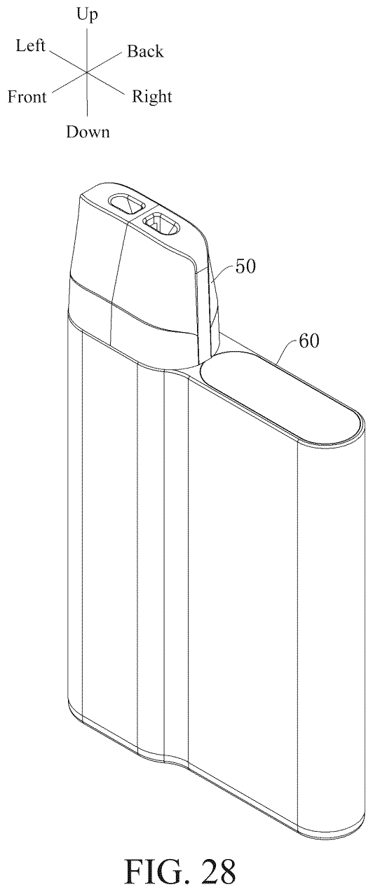

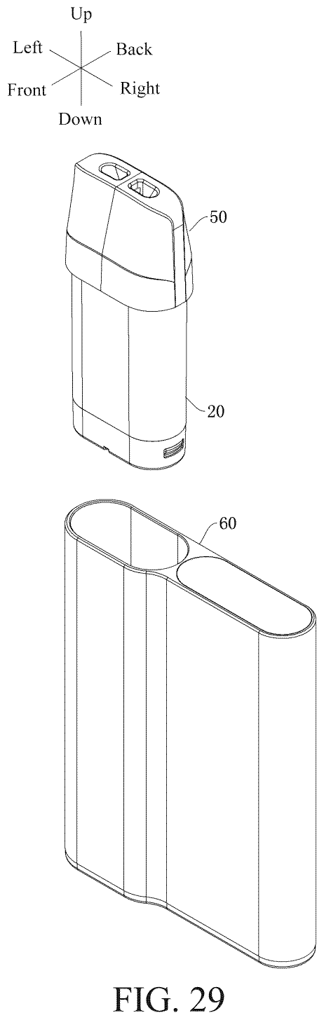

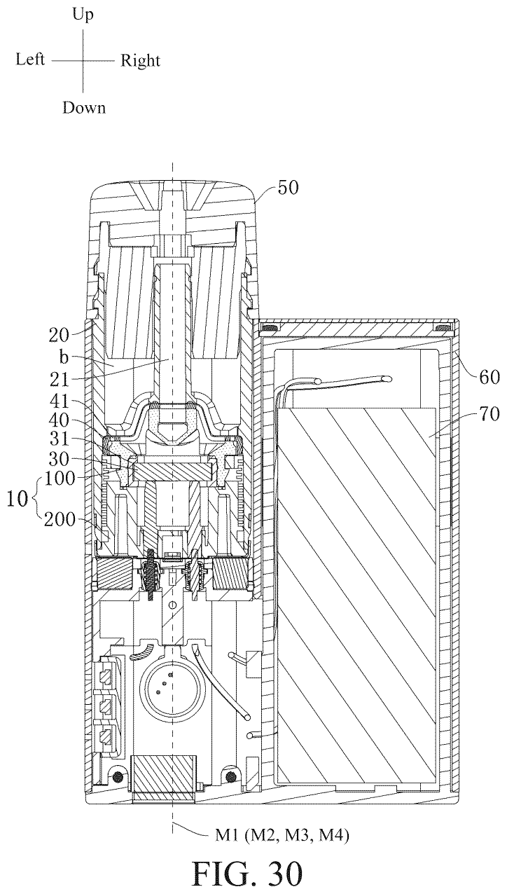

FIG. 28 is a schematic structural diagram of an electronic atomization device in an implementation of an embodiment of this application. FIG. 29 is a partial schematic structural exploded view of an electronic atomization device in an implementation of an embodiment of this application. FIG. 30 is a schematic structural cross-sectional view of an electronic atomization device in an implementation of an embodiment of this application. For ease of description, only parts related to the embodiments of this application are shown.

Based on the same inventive concept, as shown in FIG. 28 to FIG. 30, embodiments of this application provide an electronic atomization device. The electronic atomization device includes a power supply 70 and the atomizer in the foregoing embodiments, and the atomizer is detachably connected to the power supply 70. Certainly, the electronic atomization device may further include a shell 60, and the atomizer and the power supply 70 may both be accommodated in the shell 60 for ease of use by the user. In this way, because the top atomization base 100 in the foregoing some embodiments is used, the liquid flowing in the atomizer is smooth. The liquid suction process of the atomization core 30 is improved, to avoid the dry heating of the atomization core 30, thereby improving the service life of the atomizer and the user experience of a user.

In summary, in the embodiments of this application, a first corner t1 and a second corner t2 are disposed in the liquid flowing channel 120, and a position relationship between the first corner t1 and the second corner t2 is utilized to expand the outlet of the liquid flowing channel 120. Moreover, the structure of the liquid inlet opening is improved, and the liquid inlet opening is formed by different planes, so that the area of the liquid inlet opening is increased, thereby further improving liquid supply. On this basis, a boss 130 is disposed, so that the area of the liquid inlet opening can be increased, and a space above the atomization core 30 can be increased, so that liquid supply is improved, and the weight of the overall structure can be further reduced. In this way, a liquid supply space is improved, and an escape space for bubbles generated when air passes through the liquid flowing channel 120 is further enlarged, to mitigate unsmooth liquid flowing, thereby improving the service life of the atomizer, and improving the user experience of a user.

In the descriptions of this application, the descriptions of reference terms such as “in an embodiment”, “in some embodiments,” “in some other embodiments”, “in still some other embodiments”, or “examples” mean that a feature, structure, material, or characteristic that is described with reference to the embodiment or the example is included in at least one embodiment or example of the embodiments of this application. In this application, schematic descriptions of the foregoing terms are not necessarily directed at the same embodiment or example. Besides, the specific features, the structures, the materials or the characteristics that are described may be combined in proper manners in any one or more embodiments or examples. In addition, a person skilled in the art may integrate different embodiments or examples described in this application and features of the different embodiments or examples as long as they are not contradictory to each other.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomizer, comprising:
a housing provided with an accommodating cavity and an air outlet channel;
an atomization base with at least a partial structure disposed in the accommodating cavity, a liquid storage cavity configured to store an aerosol-generation substrate being defined between a top wall of the atomization base and the housing, the atomization base including an atomization cavity and at least one liquid flowing channel, the liquid flowing channel being communicated between the liquid storage cavity and the atomization cavity; and
a vent channel provided with a vent outlet, the vent outlet being communicated with the liquid storage cavity and being disposed close to the air outlet channel.

2. The atomizer of claim 1, further comprising:
an atomization core disposed in the atomization cavity, the aerosol-generation substrate in the liquid storage cavity being guided to the atomization core through the liquid flowing channel, the atomization core comprising a heating body,
wherein the vent channel has a vent inlet provided in the atomization cavity, and
wherein the vent inlet is disposed in a region of the atomization base located at a periphery of the heating body.

3. The atomizer of claim 1, wherein a plurality of vent channels are provided, and the vent channels of the plurality of vent channels are symmetrically distributed along a central axis of the air outlet channel, and/or
wherein a plurality of liquid flowing channels are provided, and the liquid flowing channels of the plurality of liquid flowing channels are symmetrically distributed along a central axis of the air outlet channel.

4. The atomizer of claim 1, wherein an air guide channel and a vent opening are opened in the atomization base,
wherein the air guide channel comprises an open end and a closed end opposite the open end,
wherein the vent opening is located across two sides of the central axis of the air guide channel in a first direction,
wherein the air guide channel is communicated with the atomization cavity through the vent opening and is communicated with the air outlet channel through the open end, and
wherein the first direction is perpendicular to the central axis of the air guide channel.

5. The atomizer of claim 4, wherein the vent outlet is provided in at least one side of the air guide channel in the first direction.

6. The atomizer of claim 4, wherein the atomization base comprises a bottom atomization base and a top atomization base,
wherein the atomization cavity is defined between the bottom atomization base and the top atomization base, and
wherein the air guide channel, the vent opening, and the liquid flowing channel are opened in the top atomization base.

7. The atomizer of claim 6, wherein the top atomization base comprises:
a top base body comprising a top wall and a side wall surrounding the top wall, the top wall and the side wall surrounding to form the atomization cavity that is independent of the liquid storage cavity and provided with an opening at an end, the liquid flowing channel being provided in the top wall; and
a boss disposed protruding from the top wall, the air guide channel being formed in the boss, the vent outlet being formed in the boss and provided close to the open end.

8. The atomizer of claim 7, wherein a peripheral wall of the top base body is formed with a first vent groove communicated with the vent outlet, and
wherein an end of the first vent groove away from the vent outlet is communicated with the atomization cavity.

9. The atomizer of claim 8, wherein the first vent groove comprises a first sub-groove communicated with the vent outlet and a second sub-groove communicated with the atomization cavity, the first sub-groove is provided in the top wall and the peripheral wall of the boss, and the second sub-groove is provided in the peripheral wall of the side wall, and
wherein an end of the first sub-groove away from the vent outlet is communicated with the second sub-groove, or the peripheral wall of the bottom atomization base is formed with a second vent groove and an end of the first sub-groove away from the vent outlet is communicated with the second sub-groove through the second vent groove.

10. The atomizer of claim 9, wherein a plurality of second vent grooves are provided, and the second vent grooves of the plurality of second vent grooves are sequentially communicated, and/or
wherein the peripheral wall of the bottom atomization base is formed with the first vent groove communicating the second vent grooves.

11. The atomizer of claim 10, wherein a plurality of second sub-grooves are provided, and the second sub-grooves of the plurality of second sub-grooves are joined to and communicated with the corresponding second vent grooves.

12. The atomizer of claim 9, further comprising:
a first seal member disposed at a top end of the top atomization base,
wherein one part of the vent channel is defined between the first seal member and the groove wall of the first sub-groove, and an other part of the vent channel is defined between the side wall of the accommodating cavity and the groove walls of the second sub-groove and the second vent groove, and
wherein a first vent hole is opened in the first seal member, the first vent hole being communicated between the liquid storage cavity and the vent outlet.

13. The atomizer of claim 6, wherein a liquid inlet opening of the liquid flowing channel, the open end, and the vent outlet are all formed at a top end of the top atomization base.

14. The atomizer of claim 13, wherein the top atomization base is formed with a third vent groove communicated with the vent outlet, and
wherein an end of the third vent groove away from the vent outlet is communicated with the atomization cavity, or the bottom atomization base is formed with a fourth vent groove communicated with the atomization cavity and an end of the third vent groove away from the vent outlet is communicated with the atomization cavity through the fourth vent groove.

15. The atomizer of claim 14, wherein the third vent groove comprises a plurality of third sub-grooves formed in a peripheral wall of the top atomization base, and the third sub-grooves of the plurality of third sub-grooves are sequentially communicated, or the peripheral wall of the top atomization base is formed with a second vent groove communicating the third sub-grooves, and
wherein the fourth vent groove is communicated with the first third sub-groove of the plurality of third sub-grooves in an airflow flowing direction, and the vent outlet is communicated with a last third sub-groove of the plurality of third sub-grooves in the airflow flowing direction.

16. The atomizer of claim 15, wherein the bottom atomization base comprises a body and a connection portion disposed protruding from the body,
wherein the bottom portion of the top atomization base abuts against the body, and
wherein the peripheral wall of the top atomization base is clamped at the connection portion.

17. The atomizer of claim 16, wherein the side wall of the atomization cavity forms one part of the fourth vent groove,
wherein a top end of the body forms the other part of the fourth vent groove,
wherein the third vent groove comprises a fourth sub-groove extending inward along the peripheral wall of the top atomization base, and
wherein the fourth vent groove is communicated with the first third sub-groove in the airflow flowing direction through the fourth sub-groove.

18. The atomizer of claim 17, further comprising a second seal member disposed at the top end of the top atomization base, and
wherein one part of the vent channel is defined between the second seal member and groove walls of the third sub-grooves, and an other part of the vent channel is defined between the top atomization base and the bottom atomization base, and
wherein a second vent hole is opened in the second seal member, the second vent hole being communicated between the liquid storage cavity and the vent outlet.

19. An electronic atomization device, comprising:
the atomizer of claim 1.
